(19) United States
(12) Patent Application Publication
(10) Pub. No.: US 2003/0055300 A1
(43) Pub. Date: Mar. 20, 2003

(54) Tubular reactor for adiabatic nitration

(76) Inventors: Andreas Chrisochoou, Koln (DE); Ralf DeMuth, Hilden (DE); Thomas Linn, Grevenbroich (DE); Paul Wagner, Dusseldorf (DE); Knud Werner, Krefeld (DE)

Correspondence Address: BAYER CORPORATION, PATENT DEPARTMENT, 100 BAYER ROAD, PITTSBURGH, PA, 15205, US

(21) Appl. No.: 10/236,567

(22) Filed: Sep. 6, 2002

(30) Foreign Application Priority Data
Sep. 10, 2001 (DE) 10144481.8
May 27, 2002 (DE) 10223483.3

(51) Int. Cl. C 07C 2 5/20; B01J 2/00

(52) U.S. Cl. 568/937; 568/939; 422/239

(57) ABSTRACT

The invention relates to an optimized tubular reactor for adiabatically mononitrating aromatics, halogenated aromatics and halogenated hydrocarbons, which tubular reactor is divided into from 4 to 12 chambers by plates which have openings and effect a pressure drop of from 0.5 to 4 bar per plate.

### BACKGROUND OF THE INVENTION

1. Field of the Invention

The invention relates to an optimized tubular reactor for adiabatically mononitrating aromatics, halogenated aromatics and halogenated hydrocarbons.

2. Brief Description of the Prior Art

Nitrations of aromatics are carried out in two liquid phases. An aqueous phase which comprises sulphuric acid as catalyst, nitric acid as reaction partner, and a further component which may be, for example, phosphoric acid, which influences the ratio of the isomers formed in nitrating toluene. An organic phase which comprises the aromatics to be nitrated, and in addition comprises portions of the nitrated aromatic which forms in the course of the reaction.

Nitrations of aromatics are carried out, for example, isothermally in loop reactors, and the aqueous phase is dispersed in the organic phase, or vice versa, at one or more points in the reactor. The two phases are circulated more than once in the loop reactor before they leave the reactor. This circulation stream and its ratio to the entrance stream dictate how often and the time frequency at which the two phases pass the dispersing points of the reactor.

It is also possible to carry out nitrations of aromatics adiabatically. This type of reaction causes the reaction mixture to heat with increasing conversion and advantageously shortens the reaction time owing to the reaction acceleration resulting from the elevated temperature. A further advantage of this type of reaction is that the high temperature of the reaction mixture can be utilized to vaporize the water resulting from the reaction.

Customarily, adiabatic nitration is carried out in a tubular reactor. Owing to the absence of backflow or circulation flow, there is a more advantageous concentration profile, compared to loop reactors, which increases the space-time yield in this reactor type. Owing to the absence of the circulation flow, the dispersing points in the tubular reactor must be arranged in succession.

EP 0 779 270 B1 describes a tubular reactor which may be used for preparing an aromatic mononitro compound. The tubular reactor comprises a tube in which twisted, tabular members are arranged in series in such a way that a front margin of a twisted, tabular member is substantially perpendicular to a back margin of the preceding member. Customarily, 50 or less of these twisted, tabular members are present in a reactor, and the preferred quantity is reported to be from 4 to 12. A disadvantage of this reactor is that the twisted, tabular members arranged therein have specialized shapes which have to be specially made for this reactor type.

EP 0 489 211 describes a jet impingement reactor for carrying out mononitrations which comprises specialized internals. These internals consist of spheres and hemispheres which are provided with openings. This reactor is intended to facilitate optimal mixing of liquid phases. Disadvantages of the reactor are that its construction is costly and inconvenient and that the internals described have to be made specially.

DE 44 10 417 A1 and DE 44 11 064 A1 describe processes for adiabatically nitrating toluenes or halobenzenes. Preference is given to carrying out the nitration reaction in a reactor which contains internals for dispersing the reaction mixture, for example perforated metal sheets. The number of dispersion steps should be 2-50. However, the specifications mentioned do not state how many internals have to be present in the reactor and which other additional conditions have to be fulfilled in order to carry out an adiabatic nitration reaction to the desired final conversion.

There was therefore a need for a simply constructed tubular reactor which can be used for adiabatically preparing mononitrated compounds. The tubular reactor should be constructed in such a way that there is a sufficient dispersion effect to carry out the nitration reaction to the desired final conversion.

### SUMMARY OF THE INVENTION

Surprisingly, a tubular reactor for adiabatically mononitrating aromatics, halogenated aromatics and halogenated hydrocarbons has been found, which is characterized in that the tubular reactor is divided into from 4 to 12 chambers by plates which have openings and effect a pressure drop of from 0.5 to 4 bar per plate.

### BRIEF DESCRIPTION OF THE DRAWINGS

FIG. 1 shows a particularly preferred embodiment of the reactor according to the invention which is divided by metal sheets provided with openings, into 7 chambers.

FIG. 2 shows a plot of conversion in % (1) against the residence time in seconds (2) as illustrated in Example 1 hereinafter.

### DETAILED DESCRIPTION OF THE INVENTION

As set forth herein, investigations have shown that the desired final conversion in a process for adiabatic mononitration depends on the dispersion effect in the reactor. In order to obtain a sufficiently high dispersion effect, plates having suitable openings are installed in the reactor according to the invention which divide the reactor into chambers. The high reaction temperatures which result from the adiabatic process operation and the aggressive feedstocks place high demands on the material of the plates. Preference is given to using a material which is inert under these conditions, and tantalum plates are more preferably used. Since this material is expensive, it is particularly important for the economic viability of the nitration process to keep the number of plates as low as possible. On the other hand, in order to ensure ideal plug flow behaviour through the reactor, a minimum number of chambers and accordingly plates have to be present.

The reactor according to the invention is accordingly split by plates into from 4 to 12 chambers, preferably from 6 to 12 chambers, more preferably from 7 to 11 chambers. The plates function as dispersing elements. In the reactor according to the invention, the plates have openings. Illustrative but non-limiting examples of the openings may be slots, punch holes or drill holes. Particular preference is given to the openings being drill holes, since they are particularly simple to produce. However, other types of opening may also be chosen. Customarily, a plate has from 10 to 25 openings, preferably from 15 to 20 openings, for a mass flow of 1 t/h.

The reactor according to the invention preferably has at least one means for feeding in the reactants at the lower end and at least one means for removing the reaction mixture at the upper end. A preferred embodiment of the reactor according to the invention has means of feeding for the organic and aqueous phases which facilitate feeding into the individual chambers located in the reactor.

As well as the number and separation of the individual dispersing elements, the dispersing energy is also significant for the dispersing effect and accordingly for the desired final conversion of the reaction. The dispersing energy is generally introduced mechanically into the reaction mixture and should, in order to reduce operating costs, likewise be as small as possible. In the case of the plates, having openings, used according to the invention, the dispersing effect within such a plate is determined by the pressure drop across this plate. For reasons of mechanical stability, the pressure drop determines the thickness of the plates and accordingly the cost thereof.

In order to obtain plug flow behaviour over the entire reactor and to avoid undesirable backflows through the plates, plates are used in the reactor according to the invention which effect a pressure drop of from 0.5 to 4 bar per plate. For adiabatically mononitrating aromatics, particular preference is given to using plates which effect a pressure drop of from 0.5 to 3 bar, and very particular preference to from 0.8 to 2 bar.

For adiabatically mononitrating halogenated aromatics and halogenated hydrocarbons, plates which effect a pressure drop of from 0.5 to 3 bar per plate are preferred, and plates which effect a pressure drop of from 0.5 to 1.2 bar, per plate are more preferred.

Preference is given to keeping the pressure drop per plate as low as possible, since providing a higher pressure drop may, for example, require a pump of higher power rating which in turn leads to higher overall process costs.

According to the invention, particular preference is given to keeping the number of chambers and accordingly also the number of plates as low as possible, since the price, for example, of a tantalum plate depends substantially on the quantity of tantalum used, i.e. the thickness of the plate. When the pressure drop per plate has to be increased owing to the low number of plates, this does not have such a strong effect on the plate price, since the plate thickness is only proportional to the square root of the pressure drop.

Adiabatic mononitration in the tubular reactor according to the invention is carried out using the reactants in a composition range described, for example, in U.S. Pat. No. 5,313,009, in EP 0 436 443 B1 or in DE 44 10 417 A1. However, other compositions are also possible.

Mononitration of halogenated aromatics is carried out using the reactants in a composition range as described in U.S. Pat. No. 4,453,027 or DE 44 11 064 A1. Other compositions are also possible in this case.

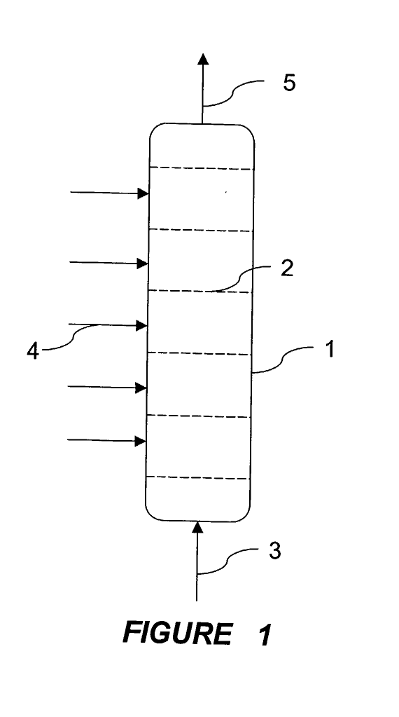

A particularly preferred embodiment of the reactor according to the invention is shown in FIG. 1, and described hereunder with reference to Figures This is a tubular reactor (1) which is divided by metal sheets (2), provided with openings, into 7 chambers. A means for feeding the reactants (3) is disposed at the lower end. Further means for feeding (4) can be used to feed reactants directly into the individual chambers. A withdrawal means (5) to let out the reaction mixture is disposed at the upper end of the reactor.

The invention is further illustrated but is not intended to be limited by the following examples in which all parts and percentages are by weight unless otherwise specified.

### EXAMPLES

### Example 1

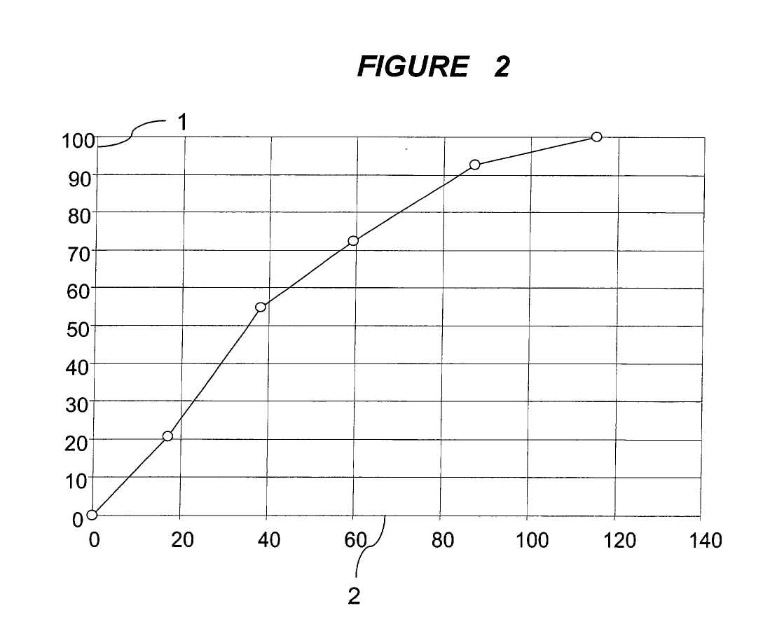

180 kg/h of approx. 70% by weight sulphuric acid were added to 8 kg/h of approx. 70% by weight nitric acid to form a 3% by weight mixed acid. This was heated in a heat exchanger to about 80° C. 9 kg/h of toluene were then admixed in a dispersion element to the mixed acid. The dispersion element was made of Hastelloy and was configured as shown in DE 199 05 572 A1. The narrowest flow cross-section on the acid side was 7 mm², on the toluene side 0.25 mm². The pressure drop on the acid side was about 0.5 bar. The dispersing element was disposed at the entrance to a thermally insulated tubular reactor (diameter 50 mm, height 3255 mm) made of enamelled steel. In the tubular reactor, a further 18 dispersion elements made of tantalum, which were configured as discs of 1 mm thickness and each provided with 4 drill holes of 1.4 mm diameter were disposed virtually evenly distributed over the total height. The pressure drop per disc was about 0.5 bar. At the downstream end of the reactor, the temperature had increased to 110° C. and all of the nitric acid had reacted. The reaction profile was determined via the temperature increase along the reactor axis (see FIG. 2). The organic and the aqueous, acidic phases are separated in a vessel at 110° C. The aqueous phase was introduced to an evaporator where the water resulting from the reaction was removed at about 90° C. A purge stream was withdrawn from the resulting reconcentrated acid in order to prevent the accumulation of by-products and replaced by fresh acid. The acid was then admixed with nitric acid again and fed back into the reactor.

In FIG. 2, the results for Example 1 are shown as a plot of conversion in % (1) against the residence time in seconds (2).

### Example 2

252 kg/h of approx. 70% by weight sulphuric acid were added to 13 kg/h of approx. 68% by weight nitric acid to form a 3% by weight mixed acid. This was heated in a heat exchanger to about 80° C. 13.9 kg/h of toluene were then admixed in a dispersion element (similar to the dispersion element for Example 1) with the mixed acid. The dispersion element was disposed at the entrance to a thermally insulated tubular reactor (diameter 50 mm, height 3255 mm) made of enamelled steel. In this tubular reactor, 4 dispersion elements were disposed which were configured as described in Example 1 and were disposed at heights of 200, 750, 1300 and 1800 mm in the reactor. Owing to the larger mass flows compared to Example 1, the pressure drop per disc was about 1 bar. At the downstream end of the reactor, the temperature had increased to 110° C. and all of the nitric acid had reacted. The rest of the procedure and the experimental set-up were similar to Example 1.

### Example 3

180 kg/h of approx. 70% by weight sulphuric acid were added to 8.3 kg/h of approx. 68% by weight nitric acid to form a 3% by weight mixed acid. This was heated in a heat exchanger to about 80° C. 8.1 kg/h of toluene were then admixed in a dispersion element similar to the dispersion element for Example 1 with the mixed acid. The dispersion element was disposed at the entrance to a thermally insulated tubular reactor (diameter 50 mm, height 3255 mm) made of enamelled steel. In the tubular reactor, there were 6 dispersion elements which were configured as discs of 1 mm thickness and each provided with 3 drill holes of 1.36 mm diameter. The dispersion elements were disposed in the reactor at heights of about 200, 500, 750, 1000, 1300 and 1800 mm. The pressure drop per disc was about 1 bar. At the downstream end of the reactor, the temperature had increased to 110° C. and all of the nitric acid had reacted. The rest of the procedure and the experimental set-up were similar to Example 1.

### Example 4

The following reaction quantities were used for the subsequent calculation: 10 t/h of toluene; 9.6 t/h of 68% by weight nitric acid; 208 t/h of 70% by weight sulphuric acid.

When the drill hole diameter d is 1.4 mm as in Example 1 and the mass flow M divided by the drill hole cross-section C is kept constant, the same pressure drop within the plate is obtained in each case as in Examples 1 to 3. For Example 1:

M_{Example 4}/C_{Example 4}=M_{Example 1}/C_{Example 1} (Equation 1)

C is obtained from the drill hole diameter d and the number of drill holes N:

C=π/4·d²·N (Equation 2)

Substituting Equation 2 into Equation 1 and solving for N_{Example 4}:

N_{Example 4}=N_{Example 1}·d²_{Example 1}/d²_{Example 4}·M_{Example 4}/M_{Example 1} (Equation 3)

The following drill hole numbers are obtained:

For Example 1:

N=4·1.4²/1.4²·(10000+9600+208000)/(8+180+9)=4621 drill holes/plates

For Example 2:

N=4·1.4²/1.4²·(10000+9600+208000)/(13+252+13.9)=3264 drill holes/plates

For Example 3:

N=3·1.36²/1.4²·(10000+9600+208000)/(8.3+180+8.1)=3281 drill holes/plates

When the reactor cross-section having a diameter D=700 mm and the drill holes are arranged in triangular pitch, the drill hole separation s can be calculated as follows:

In a triangular pitch as described, for example, in DIN 28182, the drill hole centres are located on the points of the equilateral triangle of side length s. Since all angles in such a triangle are equal to 60°, only ⅙ of each drill hole is within the triangle. Combining the drill hole areas within the triangle gives 3·⅙=½ of the drill hole area per triangle. The number of drill holes N is therefore equal to half of the number of equilateral triangles into which the reactor cross-section can be divided. The number of triangles is calculated from the ratio of the reactor cross-section to the area of a triangle:

π/4·D²/π¾·s²=√/√3·D²/s² (Equation 4)

This gives:

N=½·π/√3·D²/s² (Equation 5)

Solving for s:

s=D·√[π/(2N√3)] (Equation 6)

This gives the following drill hole separations:

For Example 1: s=700·√[π/(2·4 621√3)]=9.8 mm

For Example 2 and 3, 11.7 mm and 11.6 mm are obtained similarly.

The plate material chosen is tantalum ES according VdTÜV-Werkstoffblatt 382 (materials data sheet of the German technical surveillance association). According to this data sheet, the 0.2% extension limit R_{p0.2}=94 N/mm² at 130° C.

To calculate the necessary plate thickness, formula 19 of AD-Merkblatt B5 (German code of practice) is used. The safety coefficient is set to S=1.5 and the computation coefficient to C=0.4. Neglecting the contributions c₁ and c₂, the plate thicknesses given in the table are obtained.

| Example | Over- | Weakening | Plate | Number | Tantalum |
|---|---|---|---|---|---|
| No. | pressure | coefficient | thickness | of plates | quantity |
| 1 | 0.5 bar | 0.86 | 8.5 mm | 18 | 977 kg |
| 2 | 1 bar | 0.88 | 11.9 mm | 4 | 304 kg |
| 3 | 1 bar | 0.88 | 11.9 mm | 6 | 456 kg |

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. In an improved tubular reactor for adiabatically mononitrating a member selected from the group consisting of aromatics, halogenated aromatics and halogenated hydrocarbons, the improvement comprising the tubular reactor divided into from 4 to 12 chambers by plates which have openings and effect a pressure drop of from 0.5 to 4 bar per plate.

2. The tubular reactor according to claim 1 wherein the aromatics are benzene or toluene.

3. The tubular reactor according to claim 2 wherein the plates effect a pressure drop of from 0.8 to 2 bar per plate.

4. The tubular reactor according to claim 1 wherein the halogenated aromatics are chlorobenzene or ortho-dichlorobenzene.

5. The tubular reactor according to claim 4 wherein the plates effect a pressure drop of from 0.5 to 1.2 bar per plate.

6. The tubular reactor according to claim 1 wherein the openings are drill holes.

7. The tubular reactor according to claim 1 wherein the plates are tantalum plates.

8. The tubular reactor according to claim 1 wherein the reactor has feeding means for organic and aqueous phases which facilitate feeding into the individual chambers located in the reactor.
